(19) United States
(12) Patent Application Publication
(10) Pub. No.: US 2024/0009232 A1
(43) Pub. Date: Jan. 11, 2024

(54) COMBINATION THERAPY COMPRISING HER-2-DC1 VACCINE AND A PROBIOTIC

(71) Applicant: H. LEE MOFFITT CANCER CENTER AND RESEARCH INSTITUTE, INC., Tampa, FL (US)

(72) Inventors: Krithika N. KODUMUDI, Tampa, FL (US); Brian CZERNIECKI, Tampa, FL (US)

(21) Appl. No.: 18/021,845

(22) PCT Filed: Aug. 23, 2021
(86) PCT No.: PCT/US2021/047139

§ 371 Date: Feb. 17, 2023

(51) Int. Cl. A61K 35/15; A61K 35/742; A61K 31/337; A61K 31/519; A61K 31/506

(52) U.S. Cl. CPC A61K 35/15; A61K 35/742; A61K 31/337; A61K 31/519; A61K 31/506

(57) ABSTRACT

Disclosed are anti-cancer therapies comprising i) at least one dendritic cell pulsed with an oncodriver and ii) a fecal microbial transplant (FMT) from a pathologic complete response (pCR) donor or a cyclin-dependent kinase (CDK) inhibitor and methods of the use of said therapies to treat cancer.

### I. BACKGROUND

It is clear that infiltration of breast cancers with tumor infiltrating lymphocytes (TIL) results in greater likelihood of increased pathologic complete response (pCR) to neoadjuvant therapy and that translates to improved survival. This is further validated by the benefit that checkpoint inhibitors have in the treatment of locally advanced or metastatic breast cancer (MBC). Presence of TIL favors the response to checkpoint therapy. Response to checkpoint therapies however is limited to the minority of breast cancer patients. What are needed are new therapies that can overcome these limitations.

### II. SUMMARY

Disclosed are combination therapies comprising oncodriver pulsed dendritic cells and a fecal microbial transplant (FMT) from a pathologic complete response (pCR) donor or a cyclin-dependent kinase (CDK) inhibitor and methods of their use for treatment of a cancer.

In one aspect, disclosed herein are anti-cancer combination therapies comprising i) at least one dendritic cell pulsed with an oncodriver (such as, for example, human epidermal growth factor receptor (HER) 1 (HER1), HER2, HER3, EGFR, c-MET, B-Rapidly Accelerated Fibrosarcoma (BRAF), KIT, Androgen Receptor (AR), Estrogen Receptor (ER), KRAS, TP53, or APC) and ii) a fecal microbial transplant (FMT) from a pathologic complete response (pCR) donor (including, but not limited to an FMT that is enriched for Anaerosporobacter) or a cyclin-dependent kinase (CDK) inhibitor (such as, for example, abemaciclib, ribociclib, palbociclib, trilaciclib, or taxol). In one aspect, the oncodriver pulsed dendritic cell is activated with IL-12 prior to administration.

Also disclosed herein are anti-cancer combination therapies of any preceding aspect, further comprising at least one inhibitor of immunoregulatory molecule (such as, for example, Semaphorin (SEMA) 4D (SEMA4D), SEMA4A, SEMA4B, SEMA4C, SEMA4F, SEMA4G, SEMA3A, SEMA3B, SEMA3C, SEMA3D, SEMA3E, SEMA3F, SEMA3G, or VEGF); wherein the immunoregulatory molecule being inhibited effects the vasculature of a tumor. In one aspect, the at least one immunoregulator molecule inhibitor comprises pepinemab.

Also disclosed herein are methods of treating, inhibiting, reducing, ameliorating, decreasing, and/or preventing a cancer and/or metastasis (such as, for example, breast cancer (including triple negative breast cancer, metastatic breast cancer (MBC), ductal carcinoma in situ (DCIS), and invasive breast cancer (IBC)), melanoma, colorectal cancer, pancreatic cancer, and prostate cancer and including primary and distant tumors) in a subject comprising administering the anti-cancer combination therapy of any preceding aspect. Thus, for example, disclosed herein are methods of treating, inhibiting, reducing, ameliorating, decreasing, and/or preventing a cancer and/or metastasis (such as, for example, breast cancer (including triple negative breast cancer, metastatic breast cancer (MBC), ductal carcinoma in situ (DCIS), and invasive breast cancer (IBC)), melanoma, colorectal cancer, pancreatic cancer, and prostate cancer and including primary and distant tumors) in a subject comprising administering to the subject i) a dendritic cell pulsed with an oncodriver (such as, for example, human epidermal growth factor receptor (HER) 1 (HER1), HER2, HER3, EGFR, c-MET, B-Rapidly Accelerated Fibrosarcoma (BRAF), KIT, Androgen Receptor (AR), Estrogen Receptor (ER), KRAS, TP53, or APC) and ii) a fecal microbial transplant (FMT) from a pathologic complete response (pCR) donor (including, but not limited to an FMT that is enriched for Anaerosporobacter) or a cyclin-dependent kinase (CDK) inhibitor (such as, for example, abemaciclib, ribociclib, palbociclib, trilaciclib, or taxol).

Also disclosed herein are methods of treating, inhibiting, reducing, ameliorating, decreasing, and/or preventing a cancer and/or metastasis of any preceding aspect, wherein the wherein the oncodriver pulsed dendritic cell is administered intratumorally.

In one aspect, disclosed herein are methods of treating, inhibiting, reducing, ameliorating, decreasing, and/or preventing a cancer and/or metastasis (such as, for example, breast cancer (including triple negative breast cancer, metastatic breast cancer (MBC), ductal carcinoma in situ (DCIS), and invasive breast cancer (IBC)), melanoma, colorectal cancer, pancreatic cancer, and prostate cancer and including primary and distant tumors) of any preceding aspect, further comprising administering to the subject an immunoregulatory molecule (such as, for example, Semaphorin (SEMA) 4D (SEMA4D), SEMA4A, SEMA4B, SEMA4C, SEMA4F, SEMA4G, SEMA3A, SEMA3B, SEMA3C, SEMA3D, SEMA3E, SEMA3F, SEMA3G, or VEGF) inhibitor (including, but not limited to pepinemab).

Also disclosed herein are methods of treating, inhibiting, reducing, ameliorating, decreasing, and/or preventing a cancer and/or metastasis of any preceding aspect, wherein the dendritic cells are removed from the subject and pulsed with oncodriver ex vivo.

In one aspect, disclosed herein are methods of treating, inhibiting, reducing, ameliorating, decreasing, and/or preventing a cancer and/or metastasis of any preceding aspect, wherein the pulsed dendritic cells are administered intratumorally.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows the effect of HER2-DC1 in combination with anti-SEMA4D therapy in bilateral tumor model. Balb/C mice bearing bilateral HER+ TUBO tumors were treated with HER2-DC1 alone or antiSEMA4D or a combination of both. Combination therapy showed strong anti-tumor response with complete tumor regression in treated mice an immune subsequent challenge with TUMO cells.

FIG. 1B shows the effect of HER2-DC1+anti-SEMA4D therapy in a high tumor burden model. Complete tumor regression was observed in Her2-DC1 and anti-SEMA4D combination treated mice.

FIG. 2 shows depletion of gut microbiome by antibiotics ameliorates tumor growth in Balb/C mice bearing HER2+ TUBO tumor. Mice were treated with or without antibiotics for 14 days. After TUBO cells injection, antibiotics treated mice received antibiotics continuously until the end point. Modest increase in tumor growth was observed after continuous depletion of microbiome by antibiotics in tumor bearing mice compared to mice without microbiome depletion.

FIG. 3 shows the role of gut microbiome in response to HER2-DC1 vaccine. After 14 days of antibiotics treatment, Balb/C mice were injected with TUBO cells t the MFP. When mice had a palpable tumor, mice were divided into two groups. One group of microbiome depleted mice were received intratumoral HER2-DC1 vaccine without continuation of antibiotics treatment. Another group of mice were treated with intratumoral HER2-DC1 vaccine alone with daily administration of antibiotics

FIG. 4A shows HER2-DC1 vaccine with FMT from complete responder exhibited strong anti-tumor response. We examined the anti-tumor efficacy of intratumoral HER2-DC1 vaccine in combination with FMT from naïve, microbiome depleted TUBO bearing mice, compete responder to immunotherapy and treated non-responder mice (HER2-DC1 vaccine+anti-SEMA4D antibody in HER2+ TUBO bearing mouse model. HER2-DC1 in combination with FMT from compete responder significantly delayed tumor growth in which their gut microbiome was depleted prior to the combination treatment.

FIG. 4B shows combination treatment of HER-DC1 with FMT from responder into TUBO bearing mice that were not depleted of microbiome also showed strong anti-tumor response and 90% of treated mice had tumor regression.

FIG. 5 shows 16S rRNA sequencing of fecal samples. Fecal samples from naïve Balb/C mice, HER2+ TUBO bearing control mice, HER2-DC1 alone treated or anti-SEMA4D alone treated mice were screened for microbial abundance. In addition, fecal samples from responders or non-responders of HER2-DC1 and anti-SEMA4D antibody combination treated mice were also analyzed for gut microbiome abundance. 16S rRNA sequencing revealed differential gut microbiome abundance in non-responder mice compared to responder mice.

FIG. 6 shows 16S rRNA sequencing of fecal samples from HER2-DC1 vaccine alone or combination with FMT from complete responders. Enrichment of genus Anaerosporobacter from the phylum Firmicutes was identified in the gut microbiome of HER2-DC1 and FMT (from complete responder) combination treated mice compared to HER2-DC1 vaccine alone FIGS. 7A and 7B show anti-tumor immune response of HER2-DC1 and anti-sema4D antibody in immune deficient mice. FIG. 7A shows Balb/C Fcer1g KO mice (C.129P2(B6)-Fcer1g^{tm1Rav} N12) mice were injected at both flanks subcutaneously with 2.5×10⁵ tumor cells/site on day 0. DC were generated, matured to DC and pulsed with MHC class II new peptides. Balb/C mice received 1×10⁶/100 μl DC vaccines in left flank tumor intratumorally once a week for size weeks. Right flank tumors were left untreated. Anti-sema4D antibody was given intraperitoneally at the concentration of 10 mg/kg/body weight at weekly intervals. Control mice received isotype control antibodies, DC treatment or Mab 67 alone. Tumors were measured every 2-3 days with calipers until the endpoint. FIG. 7B shows Balb/C IFN-g KO (C.12967 (B6)-ifngtm1 Ts/J mice received 2.5×10⁵ TUBO cells subcutaneously on right flank on day 0. On day 7 when tumors were palpable, mice were randomized into four groups and treated as described above.

FIG. 8 shows anti-tumor efficacy of chemotherapy, checkpoint therapy, targeted therapies and DC1 vaccine in HER2+ BC and TNBC murine models. Balb/C mice or C57BL/6 mice received tumor cells on day 0 followed by different treatments; two doses of Taxol given intraperitoneally (i.p.) once a week; CDK inhibitor, abemaciclib was administered by oral gavage till end point; Anti-PD-L1 was given i.p. twice a week until end point; DC1 vaccine was given intratumorally.

FIG. 9A shows the efficacy of HER2-DC1 vaccine and anti-Her2 antibody combination therapy on HER2+ BC preclinical model. Balb/C mice bearing TUBO tumors were treated with intratumoral HER2-DC1 vaccine alone or anti-HER2 antibody or a combination of both. Combination treatment showed enhanced anti-tumor responses with compete tumor regression in 70% of treated mice compared to monotherapy.

FIG. 9B shows HER2-DC1 in combination with Abemaciclib (CDK inhibitor) therapy in HER2+ TUBO bearing mice model. TUBO bearing mice were treated with intratumoral HER2-DC1 vaccine alone or Abemaciclib or a combination of both. Increased anti-tumor response was observed in combination therapy received mice compared to monotherapy.

FIG. 10 shows spontaneous mammary carcinoma development in BALB-Her2/neu transgenic mice. MRI imaging was perfumed to examine the tumor development in their mammary glands at the age of week 8, week 10-11, week 12-13, and week 16.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular oncodriver pulsed dendritic cell, fecal microbial transplant (FMT), or cyclin dependent kinase (CDK) inhibitor is disclosed and discussed and a number of modifications that can be made to a number of molecules including the oncodriver pulsed dendritic cell, FMT, or CDK inhibitor are discussed, specifically contemplated is each and every combination and permutation of oncodriver pulsed dendritic cell, FMT, or CDK inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

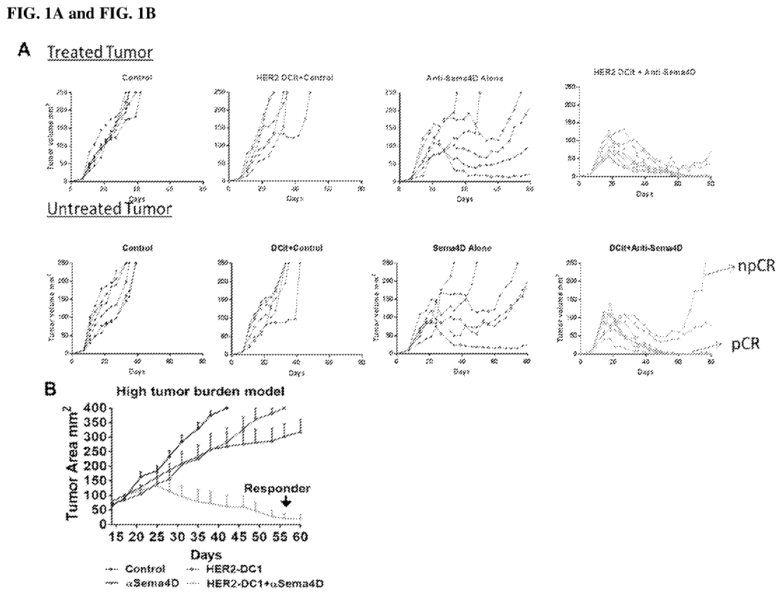

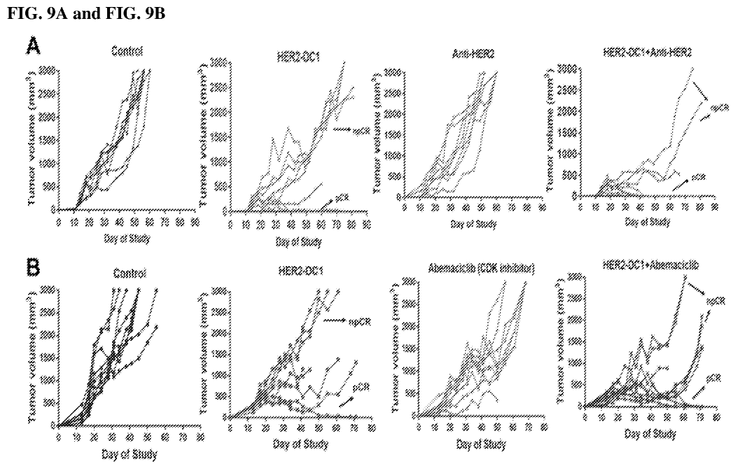

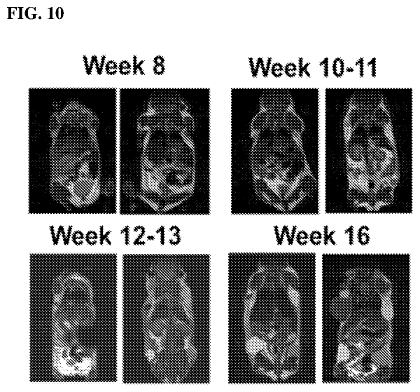

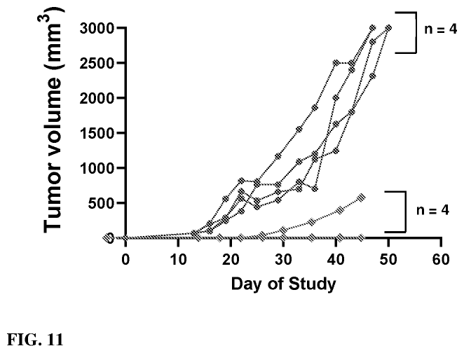

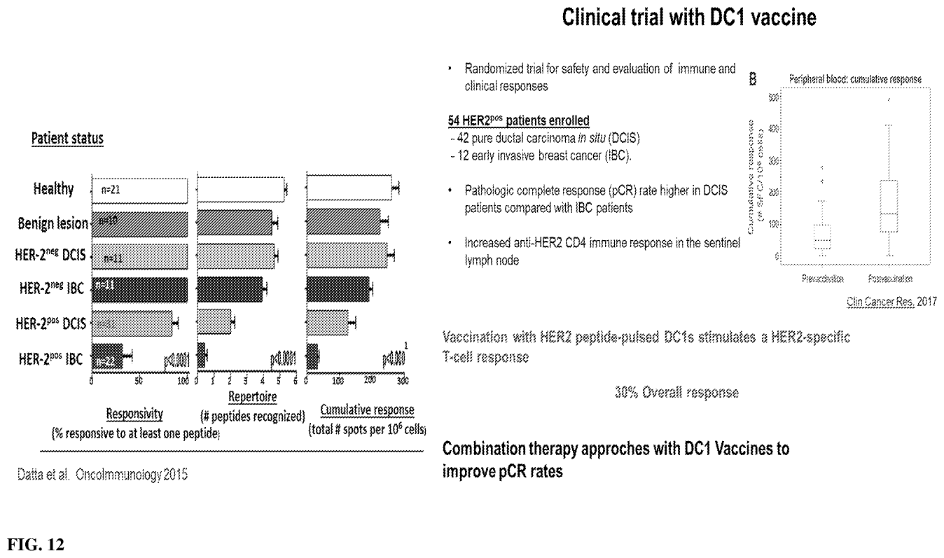

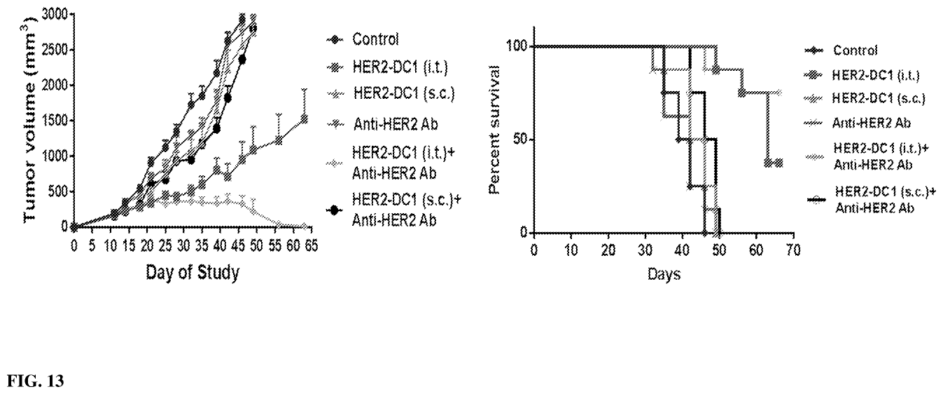

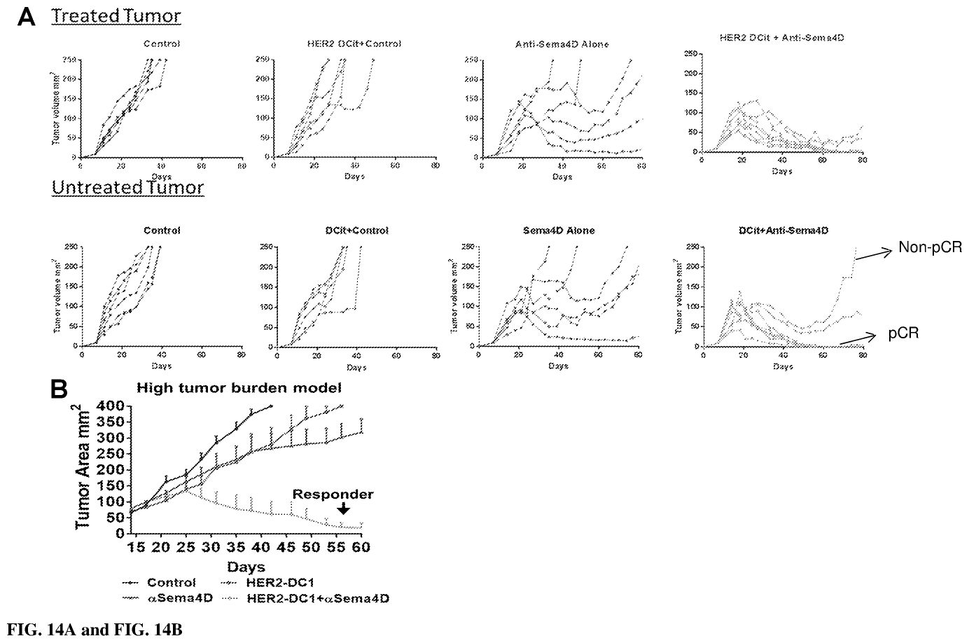

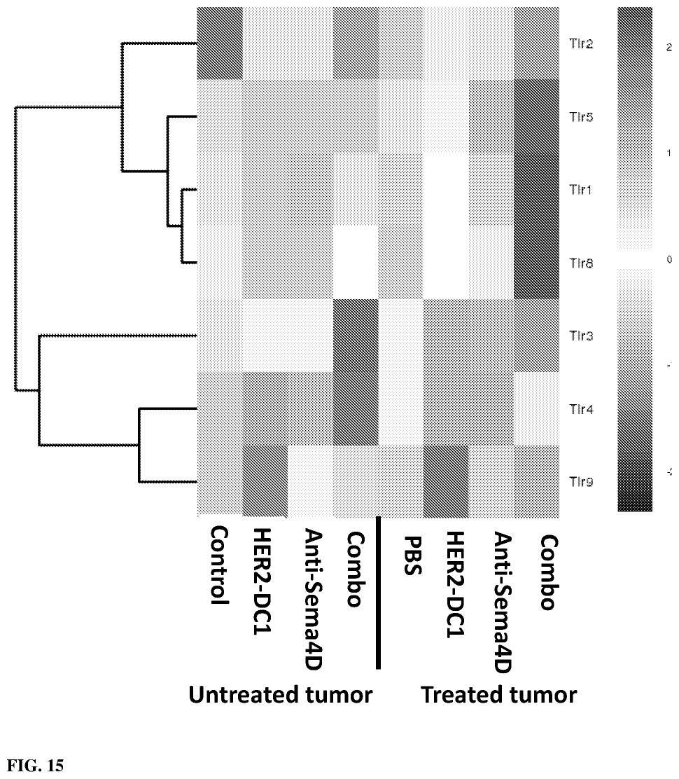

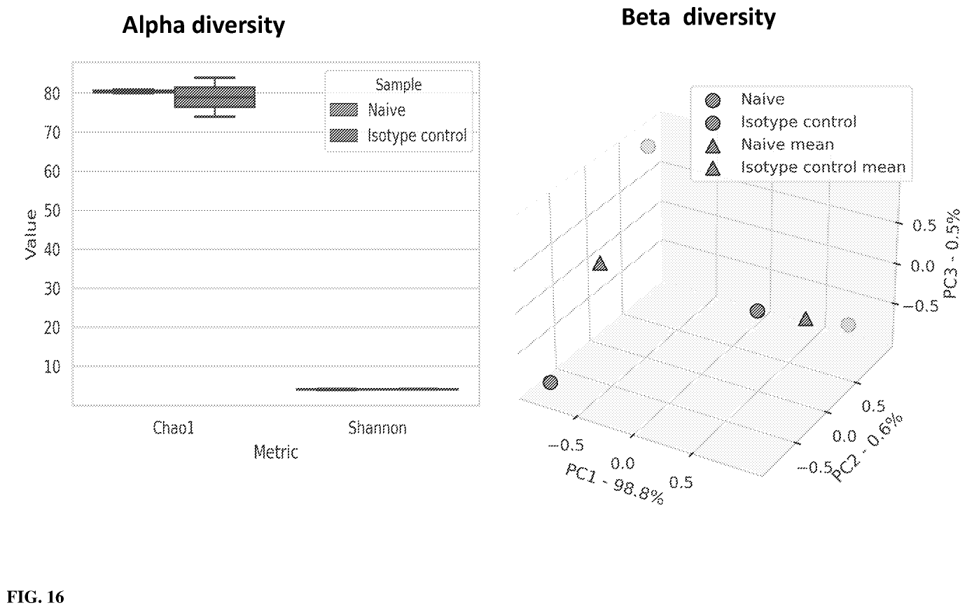

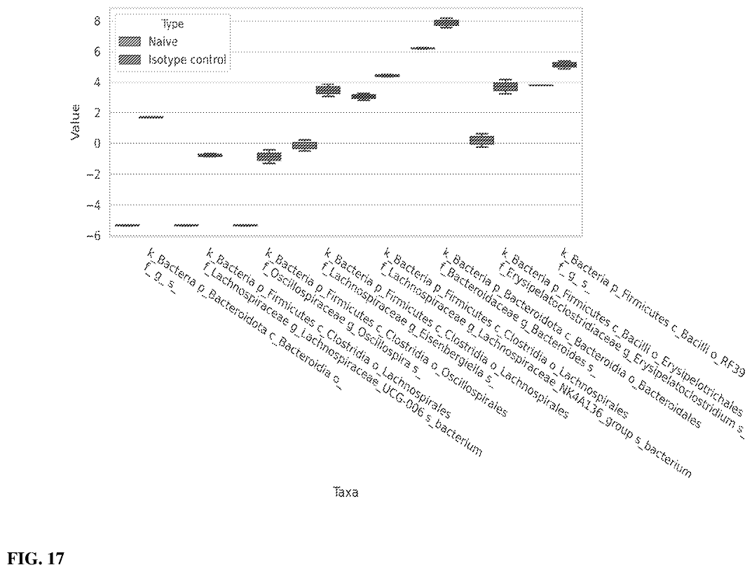

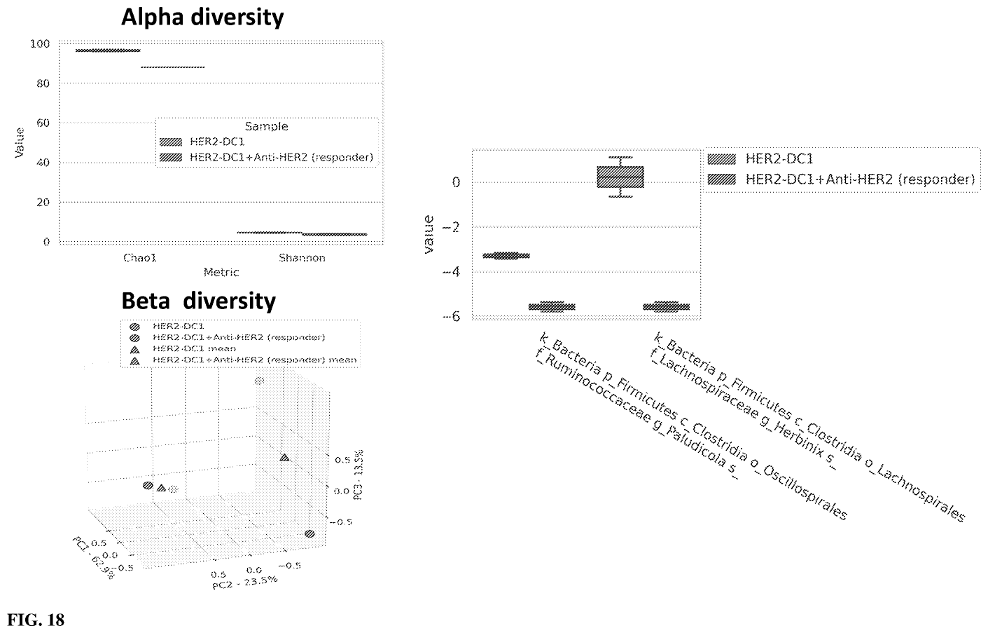

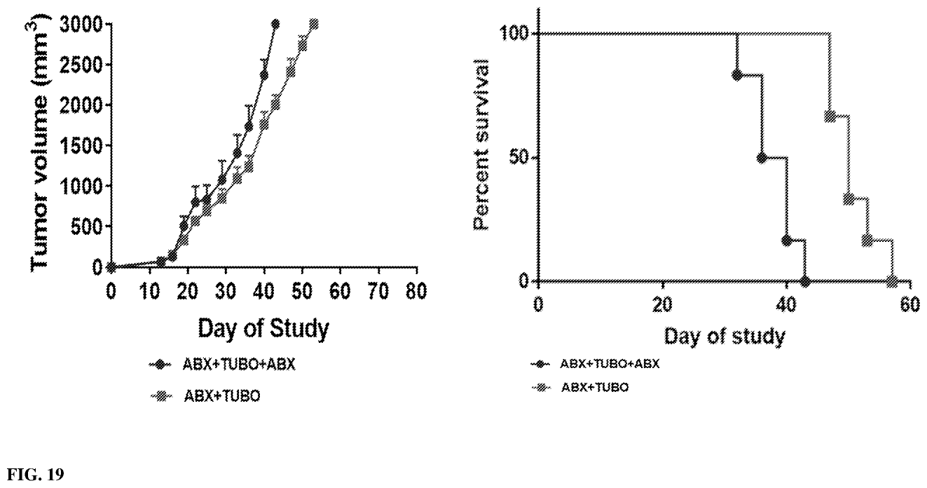

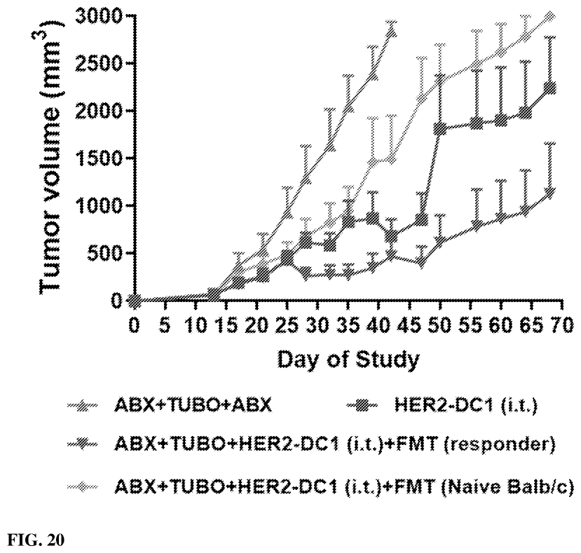

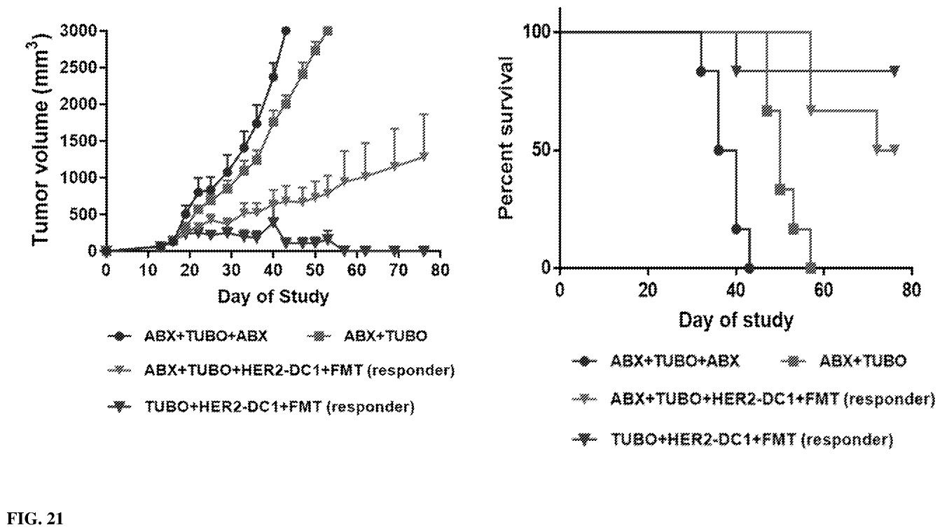

Beyond immune checkpoint therapies, there are other immune based therapies that can impact breast cancer. We have demonstrated that the anti-HER2 CD4 Th1 response is critical to the response of patients with HER2^{pos} breast cancer and is mediated through the production of interferon gamma (IFN-γ). There is a peripheral loss of anti-HER2 CD4 Th1 likely a result of migration into tumor deposits favoring positive outcome. We have demonstrated that IFN-γ works through heat shock proteins and the ubiquitin pathway to effectively increase the degradation of oncodrivers (manuscript submitted) leading to decreased stem cell markers, decreased proliferation, and enhanced induction of senescence. Systemic administration of HER2 pulsed type I dendritic cells (DC1) in DCIS and early breast cancer patients prior to surgery (neoadjuvant) results in about a 30% pCR rate. These pCR were associated with the strongest sentinel node anti-HER2 CD4 Th1 responses again reinforcing the significance of TIL migration into the tumor region. To improve upon these results, we found that intratumoral administration of the DCI with agents that increased TIL infiltration such as anti-semaphorin 4D (Pepinemab) or anti-HER2 antibodies resulted in complete regression of even large tumors in preclinical model (FIGS. 1 and 9). This therapy is successful in both HER2 and triple negative models of breast cancer and has allowed for evaluation of the gut microbiome which forms the basis of this application. Complete responses to these immune based therapies in murine models was found to dramatically alter the gut microbiome signature and impact responses.

Apart from inherited susceptibility such as BRCA mutations, several environmental factors and lifestyle components have also been strongly linked to BC. Epidemiologic studies suggest that the human microflora contributes to 16-18% or more of worldwide malignancies. Microbiota of women with breast cancer differs from that of healthy women, indicating that certain bacteria may be associated with cancer development and in response to therapy. In fact, a preexisting disturbance in human gut microbiome leads to increased breast cancer cell metastasis in a xenogeneic mouse model. Recent clinical findings suggest that antibiotic treatment in BC patients affects gut microbiome composition and correlates with cancer progression. In addition, BC patients who were on prior, or undergoing antibiotic treatment for, unrelated infections demonstrated a very poor response to conventional therapy and immunotherapy.

In melanoma, an enhanced anti-tumor response was observed following anti-CTLA-4 therapy with increased levels of microbiota including, Bacteroidales, Burkholderiales, and Clostridiales in the gut. Oral feeding of favorable microbiota such as Bacteroidales and Burkholderiales in combination with anti-CTLA-4 antibody therapy induced a strong Th1 immune response in the lymph nodes and maturation of intratumoral DCs. Anti-PD1 antibody therapy in combination with fecal microbial transplant (FMT) of favorable microbiota Bifidobacterium species improved efficacy and delayed tumor growth. In addition, Bifidobacterium species was able enhance the potential of anti-PDL1 antibody via enhancing DCs maturation and tumor specific CD8 T cells activation and decreased unfavorable gut microbiota in tumor bearing mice. Patients treated with antibiotics during anti-PD1/anti-PDL1 monoclonal antibody therapy or after the treatment had low overall survival (OS) and progression free survival (PFS) when compared to patients that did not receive antibiotics. This study also identified enriched level of Akkermansia and Alistipes microbiota in therapy responding cancer patients. The FMT enriched with Akkermansia and Alistipes (from therapy response cancer patients) into tumor bearing mice enhanced intra-tumoral infiltration of CCR9+CXCR3+CD4+ T cells in response to anti-PD1 antibody therapy. Human fecal derived Faecalibacterium transfer in a preclinical model of melanoma enhanced the anti-tumor efficacy of anti-CTLA4 antibody therapy.

In one aspect, disclosed herein are anti-cancer combination therapies comprising i) at least one dendritic cell pulsed with an oncodriver (such as, for example, human epidermal growth factor receptor (HER) 1 (HER1), HER2, HER3, EGFR, c-MET, B-Rapidly Accelerated Fibrosarcoma (BRAF), KIT, Androgen Receptor (AR), Estrogen Receptor (ER), KRAS, TP53, or APC) and ii) a fecal microbial transplant (FMT) from a pathologic complete response (pCR) donor (including, but not limited to an FMT that is enriched for Anaerosporobacter) or a cyclin-dependent kinase (CDK) inhibitor (such as, for example, abemaciclib, ribociclib, palbociclib, trilaciclib, or taxol). In one aspect, the oncodriver pulsed dendritic cell is activated with IL-12 prior to administration.

Also disclosed herein are anti-cancer combination therapies, further comprising at least one inhibitor of immunoregulatory molecule (such as, for example, Semaphorin (SEMA) 4D (SEMA4D), SEMA4A, SEMA4B, SEMA4C, SEMA4F, SEMA4G, SEMA3A, SEMA3B, SEMA3C, SEMA3D, SEMA3E, SEMA3F, SEMA3G, or VEGF); wherein the immunoregulatory molecule being inhibited effects the vasculature of a tumor. In one aspect, the at least one immunoregulator molecule inhibitor comprises pepinemab.

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHOD OF TREATING CANCER

It is understood and herein contemplated that the disclosed anti-cancer combination therapies can be administered via any route determined to be appropriate by the attending physician. Administration” to a subject includes any route of introducing or delivering to a subject an agent either locally and/or systemically. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intratumoral, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

It is understood and herein contemplated that while a single administration of the components of the disclosed anti-cancer combination therapies (i.e., the pulsed dendritic cells and/or the immunoregulator molecule inhibitor) would be ideal, not every patient will respond in the same manner. Thus, in one aspect, disclosed herein are anti-cancer combination therapies methods treating, preventing, reducing, and/or inhibiting a cancer; wherein the at least one pulsed dendritic cell, FMT, CDK inhibitor, and/or immunoregulatory molecule inhibitor is administered at least 1, 2, 3, 4, 5, 6, 7, 8, 9, 10, 11, 12, 13, 14, 15, 16, 17, 18, 19, 20, 21, 22, 23, or 24 times per day or at least 1, 2, 3, 4, 5, 6, 7, 8, 9, 10, 11, 12, 13, or 14 times per week for at least 1, 2, 3, 4, 5, 6, 7, 8, 9, 10, 11, 12, 13, 14 days, 3, 4, 5, 6, 7, 8, 9, 10, 11, 12 weeks. Also disclosed herein are anti-cancer combination therapies methods treating, preventing, reducing, and/or inhibiting a cancer: wherein the at least one pulsed dendritic cell, FMT, CDK inhibitor, and/or immunoregulatory molecule inhibitor is administered at least 1, 2, 3, 4, 5, 6, 7, 8, 9, 10, 11, 12, 13, 14, 15, 16, 17, 18, 19, 20, 21, 22, 23, or 24 times per day or at least 1, 2, 3, 4, 5, 6, 7, 8, 9, 10, 11, 12, 13, or 14 times per week for at least 1, 2, 3, 4, 5, 6, 7, 8, 9, 10, 11, 12, 13, 14 days, 3, 4, 5, 6, 7, 8, 9, 10, 11, 12 weeks. It is further understood and herein contemplated that the order and duration of the administered components can vary as appropriate for the subject being treated. In one aspect, disclosed herein are anti-cancer combination therapies methods treating, preventing, reducing, and/or inhibiting a cancer; wherein the pulsed dendritic cells are administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the FMT, CDK inhibitor, and/or immunoregulatory molecule inhibitor; are administered concurrently with the FMT, CDK inhibitor, and/or immunoregulatory molecule inhibitor; or wherein the at least one anti-cancer agent is administered at least 1, 2, 3, 4, 5, 6, 7, 8, 9, 10, 11, 12, 18, 24, 30, 36 hours, 2, 3, 4, 5, 6, 7, 8, 9, 10, 11, 12, 13, 14, 21, 28, 30, 31, 45 days, 2, 3, 4, 5, or 6 months prior to administration of the pulsed dendritic cells.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

As noted above, it is intended herein that the disclosed methods of treating, inhibiting, reducing, ameliorating, and/or preventing cancer can augmented with any therapeutic treatment of a cancer including, but not limited surgical, radiological, and/or pharmaceutical treatments of a cancer. As used herein, “surgical treatment” refers to tumor resection of the tumor by any means known in the art. Similarly, “pharmaceutical treatment” refers to the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolcvulinic Acid, Anastrozole, Aprcpitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplalin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinst (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab).

In one aspect, it is understood and herein contemplated that the pulsed dendritic cells can be activated prior to administration as well as prior to being pulsed with the oncodriver. Activation of the dendritic cells (DC1) can be achieved by contacting the cells with IFN-γ, TNFα, CD40, IL21, and/or IL-12. Accordingly, disclosed herein are anti-cancer therapies or methods of treating, preventing, reducing, and/or inhibiting a cancer or metastasis, wherein the HER-3 CD4+ T cell epitope pulsed dendritic cell is activated with IL-12 prior to administration.

In one aspect, it is further understood that the subject's own dendritic cells can be removed and pulsed ex vivo and transferred back to the subject for use in the disclosed anti-cancer combination therapies for treating, preventing, reducing, and/or inhibiting a cancer. Thus, disclosed herein are methods of treating, preventing, inhibiting, or reducing a cancer or metastasis, wherein the at least one dendritic cell is removed from the subject and pulsed with the HER-2 CD4+ T cell epitope ex vivo.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: DC1 Vaccine in Combination with SEMA4D Blockade

We have demonstrated that vaccination using type I polarized dendritic cells (DC1) drives anti-tumor CD4 Th1 responses in blood that do not always migrate effectively into tumors. However, delivery of these same DCs into the tumor directly leads to an improved response especially when either combined with effectors or agents that increase TIL in the tumor. Sema4D is a member of family of cell surface molecules that are essential for tissue and organ development and are involved in immune regulation. Antibodies against Sema4D have been shown to regulate lymphocyte infiltration into tumors. Pepinemab is a humanized IgG4 monoclonal antibody which binds specifically to the Sema4D, blocking binding to plexinB1 (PLXNB1), plexin B2 (PLXNB2), and CD72. Blockade of Sema4D appears to enhance TIL into the tumor in preclinical studies. Pepinemab is currently under investigation for use in colorectal cancer, lung cancer, and pancreatic cancer at the 10 mg/kg IV dosing every 2 weeks. In collaboration with Vaccinex, we combined anti-Sema4D antibody with intratumoral delivery of DCI in preclinical HER2 tumor models. Systemic administration of anti-Sema4D antibody combined with intratumoral HER2 pulsed DCI into one of the tumors in a dual established HER2 tumor model resulted in complete tumor regression (pCR) of both tumors in about 80% of mice compared to treatment with anti-Sema4D antibody or DC1 alone (FIG. 1A). Regression of non-injected contralateral tumors after HER2-DC1 and anti-Sema4D antibody combination therapy indicated superiority to the immune response generated. Complete tumor regressed mice were considered as complete responders (pCR) as they were immune to tumor challenge. We observed this effect even in high tumor burden preclinical mouse models (FIG. 1B). There are about 20% of mice where tumors initially regress but regrow and are referred to as non-responders (npCR).

### 2. Example 2: Depletion of Gut Microbiome Leads to Increased Tumor Growth in Preclinical Model for HER2+ Breast Cancer

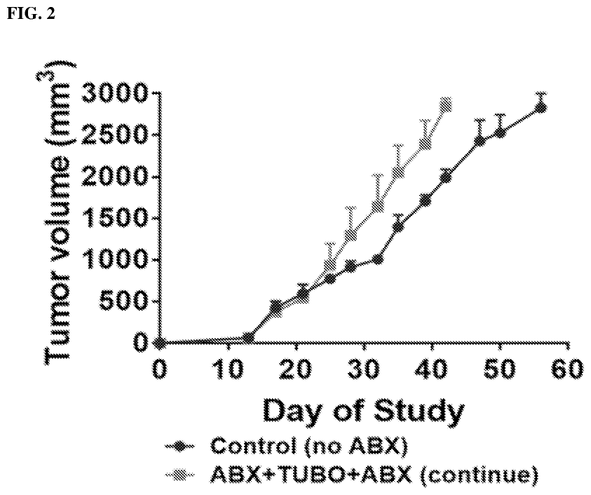

In order to understand whether depletion of gut microbiome influences HER2^{pos} BC progression, Balb/c mice were treated with or without broad spectrum antibiotics by daily oral gavage. After 14 days of antibiotics, HER2^{pos} TUBO cells (Turin-Bologna-mammary cell carcinoma derived from spontaneous BALB-neuT mice) were injected orthotopically in the mammary fat pad (MFP) of the experimental mice. Mice received antibiotics continuously every day until the end point. Tumor growth was measured every 2-3 days. We observed a modest increase in tumor growth and poor survival in mice at least partially depleted of gut microbiome by antibiotics compared to mice without antibiotic treatment (FIG. 2). This data indicates that the gut microbiome can have an impact on tumor growth.

### 3. Example 3: Role of Gut Microbiome in Response to HER2-DC1 Vaccine on Tumor Growth in a HER2^{pos} BC Mouse Model

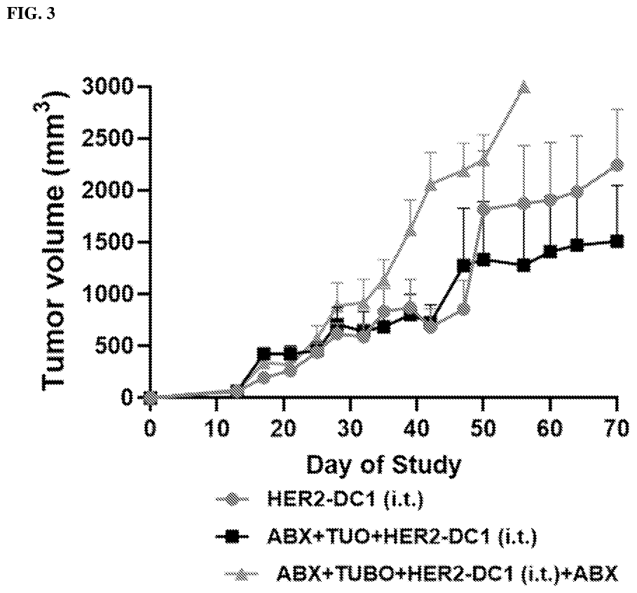

Broad spectrum antibiotics were administered orally to Balb/C mice daily for 14 days followed by injection of TUBO cells orthotopically. Mice with palpable tumors were randomized into two experimental groups. One group of microbiome depleted mice received intratumoral HER2-DC1 vaccine weekly once for 6 weeks with no additional antibiotic treatment. The other group was treated with intratumoral HER2-DC1 vaccine along with daily administration of antibiotics. As shown in FIG. 3, significant delayed tumor growth was observed after HER2-DC1 intratumoral injection in tumor bearing mice that had microbiome depletion prior to tumor induction. However, the anti-tumor efficacy of HER2-DC1 vaccine was abrogated in HER2^{pos} TUBO tumor bearing mice that were continuously treated with antibiotics. This data indicate that gut microbiome play a significant role in response to HER2-DC1 therapy.

### 4. Example 4: HER2-DC1 Vaccine in Combination with FMT from Complete Responder Exhibits Strong Anti-Tumor Response in HER2^{pos} BC

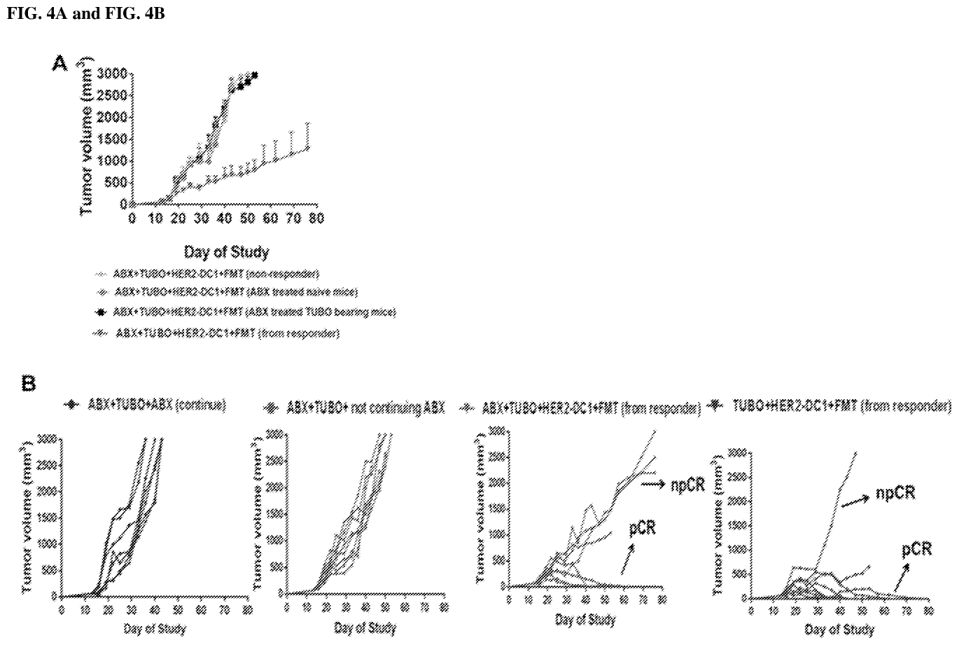

The mice cured after combination treatment of intratumoral delivery of HER2-DC1 with anti-SEMA4D antibody were immune to subsequent HER2^{pos} TUBO tumor challenge. We next examined the immune modulatory role of gut microbiome in mice achieving a pCR. The combination treatment of intratumoral HER2-DC1 vaccine with fecal microbial transplant (FMT) from naïve, microbiome depleted TUBO bearing mice, complete responder (pCR) to immunotherapy and treated non-responder (npCR) mice was carried out. DC1 treatment in combination with FMT from only the pCR mice significantly delayed tumor growth with 50% pCR achieved in mice that were previously depleted of gut microbiome compared to the control groups (FIG. 4A). We did not observe this effect in mice treated with HER2-DC1 vaccine in combination with FMT from either naïve mice, microbiome depleted TUBO bearing mice and non-responder mice (FIG. 4A). Interestingly, we observed superior anti-tumor response in mice that received combination treatment with HER2-DC1 with FMT from complete responders without any prior depletion of host gut microbiome. We observed tumor regression in 90% of treated mice (FIG. 4B). This data indicates a major contribution from the gut microbiome to partially effective immunotherapy.

### 5. Example 5: Gut Microbiome in Response to HER2-DC1 Vaccines

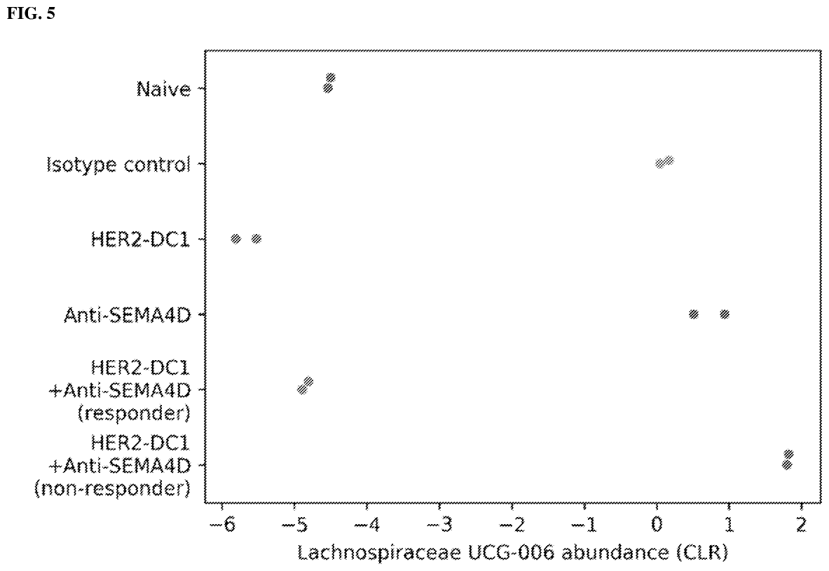

Gut microbiome compositional differences significantly influence cancer development and anti-tumor responses to conventional therapy and immunotherapy. We sought to investigate if the microbial composition differs in HER2^{POS} TUBO tumor bearing mice that received HER2-DC1 vaccine alone or in combination with anti-SEMA4D antibody. In addition, we examined the microbiome composition in mice that were complete responders or non-responders after treatment. Taxonomic profiling of fecal materials using 16S rRNA gene sequencing revealed abundances of bacterial taxa in the gut microbiome of HER2^{POS} TUBO bearing mice and anti-SEMA4D treated mice when compared to naïve mice. Further, we observed differences in several taxa, including enrichment of Lachnospiraceae UCG-006, in non-responder mice compared to responder mice (FIG. 5). Similar increases in non-responders were observed in Escherichia, Turicibacter, and Lactobacillus genera. In contrast, responders showed increased abundances in the Butyricicoccus and Bacteroides genera, and the species Butyricimonas paravirosa. Interestingly, we found low abundance of these taxa in gut microbiome of pCR mice. We propose that the abundance of these identified bacteria taxa in gut microbiome impacts HER2^{POS} BC progression and their further enrichments can induce therapy resistance. Also, we propose that HER2-DC1 vaccine and anti-SEMA4D combination therapy can reduce the level of tumor promoting bacterial taxa and enhance anti-tumor immune response in HER2^{POS} BC.

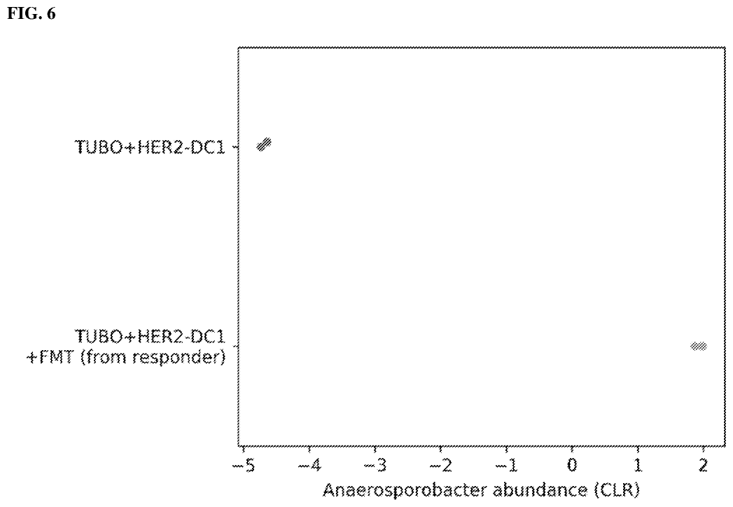

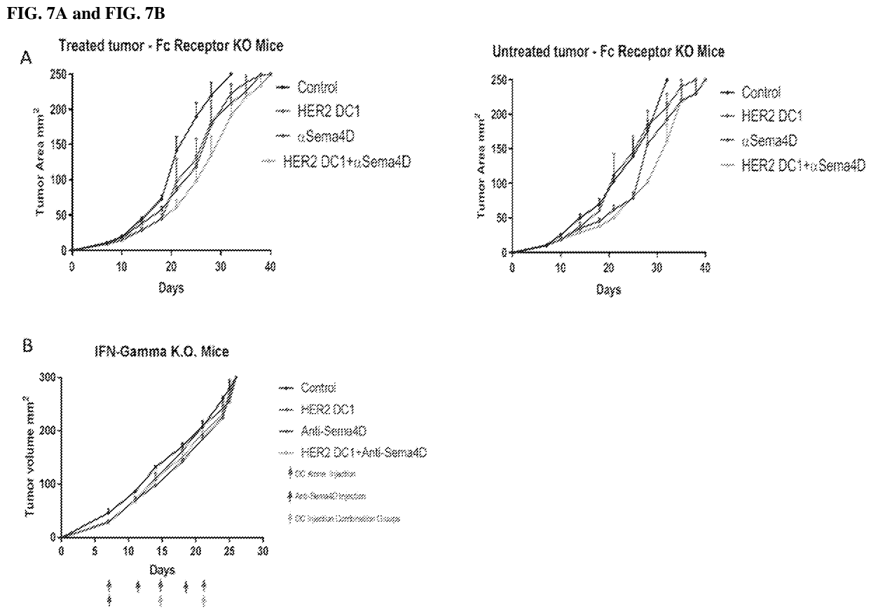

Next we investigated the bacterial composition and abundances present in the gut microbiome of HER2^{POS} TUBO tumor bearing mice that were treated with HER2-DC1 vaccine in combination with FMT from pCR. We identified higher enrichment of genus Anaerosporobacter from the phylum Firmicutes in the gut microbiome of HER2-DC1 vaccine and FMT (from pCR) combination treated mice compared to HER2-DC1 alone treatment (FIG. 6). We propose that the FMT containing enriched genus Anaerosporobacter from pCR (HER2-DC1 and anti-SEMA4D combination treated mice) can enhance the efficacy of HER2-DC1 vaccine mediated anti-tumor immune response and control HER2^{POS} BC progression. A favorable gut microbiome develops in subjects achieving a pCR to immunotherapy and that alternatively the microbiome can contribute to tumor growth and metastasis. In this proposal we can characterize the microbiome in preclinical and clinical samples, using 16S rRNA gene sequencing and selected whole shotgun metagenomic sequencing, to determine whether a favorable, unique microbial signature develops in those achieving pCR to immunotherapy. We can investigate the effect of pCR gut microbiota in preclinical models of immunotherapy in several subtypes of breast cancer, determine its role in prevention and promoting tumor growth in transgenic spontaneous tumor models, and identify the major mechanism through which the microbiome mediates this activity. In addition, we can assess in several human neoadjuvant therapy models whether patients that develop a pCR to neoadjuvant immunotherapy develop a compensatory microbiome that is immune stimulating in preclinical models and whether we can identify a microbiome that favorably impacts human breast cancer treatment and prevention.

### 6. Example 6: Investigate the Relationship Between the Gut Taxonomic Diversity and Abundances in Mammary Carcinoma and Determine how that Regulates the Immune Response and Breast Cancer Growth

Based on the findings presented herein, a difference in microbial signatures were identified in the pCR and non-pCR mice that received HER2-DC1 vaccine in combination with anti-sema4D antibody. In addition, when the fecal samples from pCR mice were transferred to HER2^{POS} TUBO bearing mice (that were initially treated with antibiotics) in combination with HER2-DC1 we observed significant delay in tumor growth with 50% having pCR. In addition, in TUBO bearing mice that received FMT from pCR mice in combination with HER2-DC1 vaccine, without prior antibiotics a strong anti-tumor response was induced with 90% pCR. We propose that commensal gut microbiota can play a role in response to immune based therapy in HER2^{POS} BC as well as other subtypes. This occurred in syngeneic mice given the same nutrition and having the same living conditions, yet there were dramatic changes in the gut microbiome signatures. The fact that we have similar subtypes of mammary carcinoma in mice, with controlled genetics and diet, offers an incredible opportunity to explore whether achieving a pCR in these models is the result of differential gut microbial signatures. In addition, we can determine whether these signatures can be identified across tumor subtypes or strains of mice so they can be developed as a therapeutic, and determine the mechanism by which the gut microbial signature impacts anti-tumor immunity. This can offer an substantial opportunity to move the field of microbiome research and its potential treatment forward in breast cancer therapy. Furthermore, the data indicates increased tumor growth in mice treated with triple antibiotics. Hence, we can determine whether the gut microbiome through an immune response also can favor tumor growth and survival in HER2^{POS} BC or TNBC murine models.

Here we can utilize two different mouse strains: —Balb/C mice which generate more Th2 like responses and C57BL/6 mice which generate strong Th1 responses as models. We identify the microbial signatures between tumor bearing and non-tumor bearing Balb/C and C57BL/6 mice using 16S ribosomal RNA (rRNA) sequencing. We can use 8-10 mice per group for each experiment.

a) Status of Gut Microbiome in HER2^{POS} BC or TNBC Mouse Models:

To investigate the status of gut microbiome on the immune response as well as BC development, we can utilize HER2^{POS} and TNBC tumor models. For HER2^{POS} BC model, TUBO cells can be injected in Balb/C mice subcutaneously or in the MFP. Naïve mice without tumor cell injection can serve as control. For C57BL/6 background, we can use EO771 cells expressing HER2 and tumors are established as described above. For TNBC models, we can use 4T1 (Balb/C) and EO771 TNBC (C57BL/6) tumor cells to induce tumors. Tumor growth can be monitored every 2-3 days. Fecal samples from the naïve or tumor bearing mice can be collected at different time points. Experimental mice can be euthanatized to collect colon tissues. Fecal samples and colonic tissues can be screened for microbial composition/species richness by 16S ribosomal RNA (rRNA) gene based sequencing of various bacterial gene amplicons and metagenomics whole genome shotgun DNA sequencing. Qiagen MagAttract PowerSoil DNA KF Kit can be used for DNA extraction. This kit uses magnetic beads to capture DNA while excluding organic inhibitors. DNA yield and quality can be determined with the Qubit® 3.0 Fluorometer (Thermo Fisher Scientific, United States) using the Qubit™ dsDNA HS Assay Kit. DNA purity can be determined via 260/280 and 260/230 ratios measured on the NanoDrop 1000 (Thermo Fisher Scientific. United States). DNA integrity can be determined by 0.6% agarose gel electrophoresis and visualized. The V4 region of 16S rRNA gene can be amplified using PCR with modified 515F and 806R primers, followed by amplicon sequencing on the MiSeq platform (Illumina, San Diego, CA) based on existing protocols to generate ˜100,000 250-bp paired-end reads per sample. All sequencing runs can employ mock communities for positive controls and water for negative controls. The DADA2 R package can be used for read processing, including quality control, ASV identification and taxonomy annotation against the SILVA database v138. Minimum required sample depth can be determined with rarefaction curves, and low depth samples can be discarded. Selected samples can be subjected to whole metagenome shotgun sequencing. In this method, the entire bacterial genomes are sequenced in parallel with much higher sequence coverage and greater depth per sample than in 16S rRNA gene sequencing. This method can detect very low abundance members of the microbiome and can be useful in further exploring unique species in the microbial signatures we discover. The routine 16S rRNA gene sequencing is done using the Illumina MiSeq, but the WGS requires a NexSeq or HISeq Illumina instrument. This experiments provides the detailed information about the status of various gut microbial signatures in healthy condition or during HER2^{pos} BC and TNBC development.

b) To Examine the Status of Microbial Composition in Spontaneous Mammary HER2^{POS} BC Model:

To investigate the status of gut microbiome during HER2^{pos} BC development, we can use BALB-HER2/neu transgenic mouse model that develops HER2^{pos} mammary carcinoma spontaneously. This model mimics most critical disease characteristic features of human HER2^{pos} BC. Fecal samples from BALB-HER2/neu transgenic (Neu T) mice can be collected at various time points of their age: 4 weeks pups (after genotyping and identifying HER2^{POS} positive strains), 8 weeks, 10 weeks (early), 12 weeks, 16 weeks, 20 weeks (advanced), 24 weeks and 26 weeks (metastatic). To determine the microbial composition throughout tumor progression, collected fecal samples can be screened for microbial composition as detailed herein.

c) Role of Gut Microbiome on Anti-Tumor Immune Response, Influencing BC Tumor Progression and Survival:

In order to examine the role of gut microbiome and how alteration in gut microbiome influences BC development and survival, we can utilize inbred syngeneic mouse models such as Balb/C, C57BL/6 and germ-free mice models. In addition, we can utilize immune deficient models, nude and RAG KO mice on these backgrounds to determine how gut microbiota in the immune deficient mice play a role in tumor progression.

d) HER2^{pos} BC Model:

To examine the role of gut microbiome alteration during HER2^{pos} BC progression, Balb/C mice can be treated with or without daily administration of antibiotics for 14 days to deplete gut microbiome followed by HER2^{pos} TUBO cells injection at the MFP. Mice can be oral gavaged with 100 ul of antibiotics solution containing ampicillin (400 ug/ml), neomycin (200 ug/ml), vancomycin (50 ug/ml) and metronidazole (200 ug/ml). When tumors are palpable, mice can be randomized to experimental groups with following treatment conditions: 1) no initial antibiotics treatment+TUBO tumor; 2) no initial antibiotics treatment+TUBO tumor+antibiotics treatment; 3) 14 day antibiotics+TUBO tumor+antibiotics treatment follow up until end point; 4) 14 day antibiotics+TUBO tumor+no antibiotics follow up. Fecal samples can be collected at different time point. Experimental mice can be euthanatized to collect whole colonic segments. The collected fecal samples and colonic intestinal tissues can be analyzed for microbial composition as detailed herein. This study can identify the altered gut microbial signatures and their tumor promoting effects in HER2^{pos} BC.

For HER2 overexpressing EO771 tumor model, C57BL/6 mice can be administrated with antibiotics as described above followed by HER2 overexpressing EO771 cells injection and when tumor are palpable, mice can be randomized in to experimental groups as described herein. Fecal samples can be collected at different time point and analyzed for microbial composition as detailed herein. This experiment can provide insights on the role of microbiome alterations during HER2^{pos} BC progression. This Th1 dominant strain can be compared to Balb/C strains a Th2 dominant strain to compare the microbiome signatures.

e) TNBC Models:

To examine the role of gut microbiome alteration during TNBC progression we can use 4T1 (Balb/C) and EO771 (C57BL/6) tumor cell lines to establish tumors. Mice can be treated with antibiotics to deplete gut microbiome as described above followed tumor cell injection and can be randomized into experimental groups as described above. Fecal samples can be collected at different time point and analyzed for microbial composition as detailed herein.

f) Germ Free Mouse Models:

To examine the breast cancer tumor development in the absence of microbiome, we can use germ-free mouse models provided by the gnotobiotic facility of University of North Carolina (UNC). Germ-free mice are bred in isolators which fully block exposure to microorganisms, with the intent of keeping them free of detectable bacteria, viruses, and eukaryotic microbes. Germ-free mice can allow for study of different subsets of breast cancer development in the complete absence of microbes. For these experiments, we can use Balb/C and C57BL/6 germ-free mice for murine studies were Germ-free Balb/C mice can be injected with TUBO cells at the MFP or 4T1 cells subcutaneously. Germ free C57BL/6 mice can be injected with EO771 cells or HER2 overexpressing EO771 cells subcutaneously. Balb/C mice bearing HER2 TUBO tumors or 4T1 tumors or C57BL/6 mice bearing EO771 tumors that are not depleted of microbiome can be used as controls respective of tumor models. Tumor progression can be monitored for every 2-3 days between tumor bearing Balb/C mice that are not depleted of microbiome and tumor bearing germ free mice. Fecal samples can be collected at different time point and analyzed for microbial composition as detailed herein. Studies described herein address how altered or absence of gut microbiome influences HER2^{pos} BC and TNBC progression and survival.

g) Reciprocal Interaction Between Microbiome and Immune Responses:

Increasing evidence indicates that microbiome can also influence peripheral immune cell populations, providing a mechanism by which microbes influence disease pathology. In addition, multiple studies support that gut microbiome can profoundly influence the potency of immunotherapy and some chemotherapy with immunostimulatory functions. Another study has shown depletion of Foxp3+regulatory T Cells was accompanied by an increase in the relative abundance of Firmicutes in the murine gut microbiome. Studies presented herein investigated what immune mechanism is driving anti-tumor immune response and complete tumor regression in TUBO bearing mice treated with HER2-DC1 and anti-sema4D combination therapy, we used Balb/C IFN-γ KO (C.129S7 (B6)-Ifng^{tm1Ts}/J (IFN-γKO) and FcR BALB/C-Fcer1g KO mice (C.129P2(B6)-Fcer1g^{tm1Rav} N12) mice. We investigated whether Fc gamma receptor (FcγR) which is necessary for antibody dependent cellular cytotoxicity (ADCC) mechanism, is required for tumor regression induced by combination treatment with anti-SEMA4D antibody and HER2-DC1 vaccine. Natural killer (NK) cells are the principal cell type involved in this ADCC mechanism. NK cells are known to express FcγR which binds to the Fc portion of IgG (immunoglobulin G) antibodies that bounded on the surface of tumor cells. This event mediates cytotoxic activity of NK cells to lyse targeted tumor cells and further NK cells mediated secretion of Th1 cytokines (IFN-γ and TNF-α). FcγR expression is also involved in activating various phases of immune responses, importantly adoptive immune response by regulating DC and B cells activation and innate immune response by triggering innate immune effectors cells. BALB/C-Fcer1g KO mice (C.129P2(B6)-Fcer1g^{tm1Rav} N12) mice were injected at both flanks subcutaneously with TUBO cells on day 0 followed by HER2-DC1 vaccine as described above with only one tumor receiving intratumoral HER2-DC1 injections and the untreated tumors were injected with saline. As shown in FIG. 7A, although combination treatment delayed tumor growth in FcγR-deficient mice, no complete tumor regression was observed. To investigate whether the complete tumor regression induced by combination therapy is IFN-γ mediated, we used a single tumor model, Balb/C IFN-γ KO (C.129S7 (B6)-Ifng^{Tm1Ts}/J mice were injected with TUBO cells subcutaneously on right flank on day 0. When tumors were palpable, mice were randomized into four groups. Mice received monotherapy with either control antibody or anti-sema4D antibody (10 mg/kg/body weight) intraperitoneally until end point or 1×10⁶/100 ul intratumoral HER2-DC1 weekly for six weeks. For combination therapy, mice received anti-sema4D antibody prior to receiving first intratumoral HER2-DC1 injection once a week for six weeks. For combination therapy, mice received anti-sema4D antibody prior to receiving first intratumoral HER2-DC1 injection once a week for six weeks. As shown in FIG. 7B, monotherapy or combination therapy failed to induce anti-tumor immune response in the absence of IFN-γ and the combination therapy efficacy was completely abrogated in the IFN-γ KO mice indicating the anti-tumor immune response was mediated by IFN-γ. These data indicate that the antitumor effects of combination therapy are mediated through immune mechanism. Based on these findings, we can examine the reciprocal interaction between the gut microbiota and host immune response during tumor progression and how gut microbiome and immune system influence each other. To address this we can utilize, immune deficient mouse models.

h) Immune-Deficient Models:

To investigate how the gut microbiota alters host immunity and its role in breast cancer tumor progression, we can utilize nude and RAG KO mice that are deficient in adaptive immunity and lack mature lymphocytes. Fecal samples can be collected from these mice prior to tumor injection. We can utilize nude or RAG KO immune deficient mice with Balb/C or C57BL/6 background. For HER2^{POS} BC model, nude or RAG KO Balb/C can be injected with or without HER2^{POS} TUBO cells at the mammary fat pad (MFP). For 4T1 TNBC model, nude or RAG KO Balb/C mice can be injected subcutaneously with or without 41′1 cells. For EO771 TNBC model, we can inject EO771 cells subcutaneously into nude or RAG KO C57BL/6 mice. Tumor growth can be monitored every 2-3 days. Fecal samples can be collected to investigate the gut microbial diversity and this can provide evidence whether lack of adaptive immunity can alter the composition and diversity of gut microbiota in turn can modulate the immune response leading to tumor progression. In addition, changes in gut microbiota from normal to immune-deficient mice can be investigated by 16S rRNA amplicon-based sequencing as detailed herein, which verifies whether significant gut microbiota shifts occur in immune deficient mice and its role in tumor progression.

i) Flow Cytometry and Immunophenotyping:

The inter-link between gut microbiome and the status of various immune cells during HER2^{pos} BC or TNBC development can be examined. In addition, how altered or absence of specific gut microbiome affect host immune response and its role in creating immunosuppressive tumor microenvironment during HER2^{pos} BC or TNBC progression can also investigated. We can perform a comprehensive analysis of T cells and suppressor cell populations in the spleen, lymph nodes (LNs), and tumors. T cells can be measured by staining for CD3, CD4, and CD8. T cells activation markers can include CD28, CD44, CD62L and CCR7. Proliferation and effector function can be measured in T cell populations by staining for Ki67, granzyme B and IFN-γ and analyzed by flow cytometry. Individual suppressor cell populations can be measured including CD11b+Gr-1+ myeloid derived suppressor cells (MDSC) and CD4+CD25+Foxp3+ regulatory T cells (Tregs). Tumor-Associated Macrophages (TAMS) can be identified using the M1 markers including F4/80, CD80, MHC class I and II, TLR2 and TLR4 and M2 markers including CD163, arginase, β-glucan (Dectin-1) and mannose receptor (CD206). In addition, paraffin-embedded tumor tissue samples can be stained for lymphoid and myeloid subsets by immunohistochemistry. This work provides an extensive evidence of modulatory role of gut microbiome on host immune response and in mediating immune suppressive microenvironment in HER2^{pos} BC and TNBC.

### 7. Example 7: Determination Whether pCR to any Therapy Results in Similar Changes in Microbiome in Different Mouse Strains with Different Subtypes of Mammary Carcinoma

a) Anti-Tumor Efficacy of Chemotherapy, Checkpoint Therapy, Targeted Therapies and DC1 Vaccine Preclinical Murine Models of Breast Cancer:

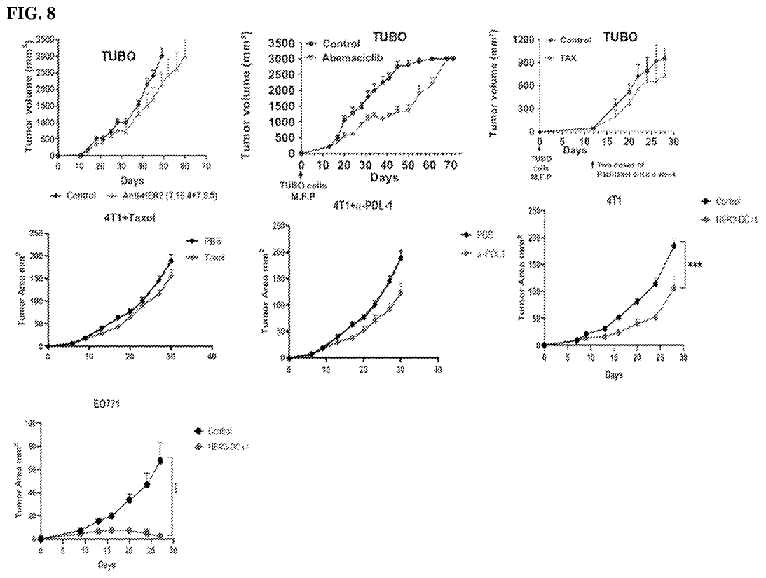

Findings presented herein have demonstrated the therapeutic efficacy of chemotherapy, checkpoint therapy and other targeted therapies in HER2^{POS} and TNBC murine models. As shown in FIG. 8. Balb/C mice bearing TUBO tumors were treated with anti-HER2 antibody (7.16.4+7.9.5) or with CDK inhibitor, abemaciclib or taxol modestly delayed tumor growth. In TNBC model, 4T1 checkpoint therapy with anti-PD-L1 significantly delayed tumor growth while treatment with chemotherapy, paclitaxel had no effect on tumor growth. Intratumoral delivery of HER3-DC1 in TNBC models, 4T1 significantly delayed tumor growth while EO771 tumor bearing mice that received HER3-DC1 had complete tumor regression. These results raise the question whether gut microbiome play a role in response to different therapies.

b) Modulatory Effect of Gut Microbiome on Chemotherapy in Preclinical Murine Models of Breast Cancer:

The status of gut microbiome in response to chemotherapy in HER2^{pos} BC or TNBC can be investigated. We can use HER2^{POS} and TNBC tumor models as described herein. When tumors are palpable, mice can be randomized to experimental groups with following treatment conditions: 1) control; 2) Paclitaxel; 3) Docetaxel. Paclitaxel or Docetaxel can be administered intraperitoneally weekly once for two weeks. Tumor growth can be monitored every 2-3 days. Survival rate of the experimental mice can also be recorded. Fecal samples from complete responders or non-responder mice can be collected at different time points. After treatments, mice can be euthanatized to collect colonic tissues. Fecal samples and colonic tissues can be screened for microbial composition/species richness as herein. These experiments allow for the identification of the status of gut microbial abundance in response to chemotherapy in HER2^{pos} BC or TNBC.

c) Modulatory Effect of Gut Microbiome in Response to Targeted Therapies in BC Murine Models:

To examine the gut microbial abundance and enrichment in response to various targeted therapies in HER2^{pos} BC or TNBC murine models, we can utilize HER2^{pos} BC of both Balb/C mice and C57BL/6 background as described herein. When tumors are palpable, mice can be randomized to following groups: 1) control; 2) anti-Sema4D; 3) anti-HER2; 4) Lapatinib; 5) CDK inhibitors. Anti-SEMA4D or anti-HER2 antibody can be administered intraperitoneally once a week for six weeks. Mice can receive oral gavage with CDK inhibitors weekly twice for six weeks. Lapatinib can be administered orally weekly once for two weeks. For TNBC models, mice can be randomized to experimental groups with following treatment conditions: 1) control; 2) anti-Sema4D; 3) CDK inhibitors. Tumor growth, survival and fecal sample collection can be done as described above. This study can address the impact of gut microbiome and their abundance after targeted therapy in HER2^{pos} BC and TNBC.
d) Modulatory Effect of Gut Microbiome in Response to Checkpoint Therapy in BC Murine Models:

The gut microbiome has been identified to influence therapeutic efficacy of immune checkpoint therapy in cancer. Here we can investigate the abundance and enrichment of gut microbiome upon immune checkpoint therapy in HER2^{pos} and TNBC models. Tumor bearing mice can be randomized to experimental groups with following treatment conditions: 1) control; 2) anti-PD-L1; 3) anti-PD-1. PD-1 or anti-PD-L1 antibody can be given intraperitoneally weekly twice for six weeks. Tumor growth, survival and focal sample collection can be done as described above. This experiment allows for the identification of the microbial abundance and enriched microbial signatures in response to immune checkpoint therapy.

e) Gut Microbiome in Response to Immunotherapy in HER2^{POS} BC or TNBC Mouse Models:

We can investigate the efficacy of gut microbiome in response to DC1 vaccine in HER2^{pos} BC or TNBC. HER2^{pos} TUBO bearing Balb/C mice or HER2 over expressing EO771 bearing C57BL/6 mice can be randomized to experimental groups with following treatment conditions: 1) control; 2) HER2-DC1. 4T1 bearing Balb/C mice or EO771 bearing C57BL/6 mice can be randomized to experimental groups with following treatment conditions: 1) control; 2) HER3-DC1. HER2-DC1 vaccine or HER3-DC1 vaccine can be administered intratumorally once a week for six weeks. Tumor growth can be monitored every 2-3 days. Survival rate of the experimental mice can also be recorded. Fecal samples can be collected as described above. The experimental outcome can identify the gut microbial composition and specific bacterial species in response to DC1 vaccine in HER2^{pos} BC or TNBC.

f) Gut Microbiome in Response to Combination Therapy in HER2^{POS} BC or TNBC Mouse Model:

Here, we can investigate the role of gut microbiome in response to combination therapy. We demonstrated the therapeutic efficacy of HER2-DC1 vaccine in combination with HER2 targeting antibodies or CDK inhibitor, abemaciclib in HER2^{pos} TUBO bearing tumor model. Balb/C mice bearing TUBO tumors were treated with intratumoral HER-DC1 alone or anti-HER2 antibody (7.16.4+7.9.5) alone or combination of both. While CDK inhibitor, abemaciclib was given daily by oral gavage alone or in combination with intratumoral HER2-DC1. We observed superior anti-tumor efficacy with for combination treatments in controlling tumor growth compared to monotherapy (FIG. 9). The intratumoral HER-DC1 with anti-HER2 antibody combination therapy resulted in complete tumor regression in 70% of treated mice and these mice were immune to TUBO cells challenge (FIG. 9A), while combination of CDK inhibitor with HER2-DC1 resulted in 30% pCR (FIG. 9B). Based on these results, we can evaluate the gut microbiome in response to different combination therapies and how different the microbial signatures are in the pCR and npCR of different combinatorial approaches to induce superior anti-tumor immune responses.

g) Tumor Re-Challenge of Complete Responders in the Presence or Absence of their Gut Microbiome:

To investigate whether gut microbiome have a role in inducing long-lasting anti-tumor immune response, mice with complete tumor regression can be re-challenged with HER2^{POS} TUBO cells or 4T1/EO771 TNBC cells respective of tumor models. Mice that reject tumors after re-challenge can be treated with antibiotics to deplete their gut microbiome and can be again re-challenged with TUBO cells or 4T1/EO771 cells. Mice without antibiotic depletion can also be monitored for tumor growth. These studies can address the crucial role of gut microbiome in generating systemic immune response against HER2^{POS} BC or TNBC

h) Role of Gut Microbiota in Modulating Cytokine/Chemokine Signatures:

Recent study has shown that gut microbiome greatly influences the production of various cytokines and chemokines (Schirmer M et al. Cell 167.4:1125-1136). Importantly, high abundance of unfavorable microbiome creates immunosuppressive tumor microenvironment via mediating immunosuppressive cytokines production in cervical cancer (Audirac-Chalifour A et al., PloS one 11.4). To investigate whether alteration in the gut microbial composition affects cytokine/chemokine signatures in the tumor microenvironment, we can collect tumor tissues and whole colonic epithelial cells from tumor bearing mice. Ex vivo tumor cells and intestinal epithelial cells can be cultured in vitro and culture supernatants can be collected and tested for Th1/Th2/Th I7/Treg cytokine/chemokine secretion by multiplex cytometric bead array. This study provides the significant correlations between gut microbial abundances and cytokine responses to stimulations during tumor progression.

i) Analysis of Microbial Signatures:

Linear models can be used to identify signature differences in microbial composition and diversity across responders and non-responders. Sequencing quality can be assessed using FastQC and MultiQC. Reads can be trimmed and sequencing adapters removed with TrimGalore, a cutadapt wrapper. Metagenomic microbial abundances can be estimated using Metagenomic Phylogenetic Analysis v2.0 (MetaPhlAn), a marker-based tool for profiling microbial communities using metagenomic sequencing. 16S rRNA amplicon sequence variants (ASVs) can be identified using DADA2 and annotated against the SILVA v138 database. The metabolic potential of the microbiome and its members can be profiled with the Human Microbiome Project (HMP) Unified Metabolic Analysis Network v2.0 (HUMAnN2), providing a functional assessment through the accumulation of gene abundances. Alpha (within group) diversity can be assessed for both richness (species counts. Chao1 score) and evenness (Shannon index), with significant differences in mean values identified using ANOVA. After applying a log-ratio transformation to account for compositionality and remove spurious correlations within the dataset, differences in beta (between group) diversity can be assessed with permutational multivariate analysis of variance (PERMANOVA). Microbial variability and association with clinical variables can be assessed with simple regression. Generalized estimating equations can be used to assess significance of log-ratio transformed species/gene/metabolite abundances and the influence of covariates across response status. The Benjamini-Hochberg method can be used to correct for multiple testing while maintaining a false discovery rate below 10%. Correlations between clinical data and covariates can be considered, with linear models adjusted accordingly. Multi-omics integration can be performed with Data Integration Analysis for Biomarker discovery using a Latent components (DIABLO), an analysis framework in the mixOmics R package that uses a variation of partial least squares regression to identify highly correlated multi-omics signatures discriminating across a categorical grouping, like response status.

### 8. Example 8: Determine Whether the Microbial Signature in Complete Responders is Independent of Genetics or Tumor Subtype can Enhance the Anti-Tumor Immune Response in Mammary Carcinoma

### Rationale:

The taxonomic profiling of fecal samples from pCR and npCR mice using 16S rRNA sequencing revealed differential microbial compositions (see FIGS. 5 and 6). We propose in that there are microbial signatures that enhance or drive an anti-tumor immune response. Here, we can identify specific microbial signatures and transfer them to tumor bearing mice to investigate their role in regulating anti-tumor immune response to breast tumor growth. In addition, we can screen for microbial metabolomics from FMT of responders in enhancing anti-tumor response in HER2^{POS} BC or TNBC murine models. We can also examine the status of oncodriver dependent signaling, TLR signaling, Th1 cytokine downstream signaling, EMT/stem cell signaling and senescence and apoptosis markers post FMT transplant.

### Approach:

a) Fecal Microbial Transplant in HER2^{POS} BC and TNBC Murine Models:

Here, we can examine FMT in syngeneic and germ free Balb/C and C57BL/6 mice. Experimental groups can receive antibiotics to deplete the host gut microbiome as described herein followed by tumor cell injection. Mice can be randomized into experimental groups with the following treatment conditions: 1) control; 2) FMT from responders; 3) FMT from non-complete responders. Mice without depletion of gut microbiome using antibiotics can be included in the study to identify if depletion of unfavorable microbiome is needed prior to the transfer of favorable microbial signatures. Fecal slurry can be prepared by suspending fecal samples in sterile PBS and filtered through 100 m strainer. 100 ul of cleared supernatant can be gavaged into mice weekly once for six weeks. Tumor growth and survival can be monitored. In addition, these experiments can be performed in germ free mice. Fecal samples can be collected at different time points from both experiments. At the end of the treatments, mice can be sacrificed by euthanasia and various organs (tumors, spleen, lymph node and whole intestine) and blood can be collected for further functional assays.

b) To Examine if the Role of Specific Microbial Signatures in Regulating Anti-Breast Cancer Immune Response:

To determine whether specific microbial signatures can improve anti-tumor efficacy independent of immune activation, CD4 T cells or CD8 T cells, immune cells depletion experiments can be performed. HER2^{POS} TUBO bearing mice or 4T1/EO771 bearing mice be randomized to experimental groups with the following treatment conditions respective of tumor models: 1) control+CD4 depletion; 2) FMT from responders+CD4 depletion; 3) FMT from non-responders+CD4 depletion; 4) control+CD8 depletion; 5) FMT from responders+CD8 depletion; 6) FMT from non-responders+CD8 depletion. Tumor growth can be monitored every 2-3 days and survival rate can be recorded. Fecal samples can be collected from all groups and the microbial diversities and abundances can be screened as detailed herein. In addition, human and murine breast cancer cell lines can be used to challenge nude mice orthotopically or into the flank to examine the direct effects of gut microbiome on cancer cells, independent of immune activation. To address this, nude mice can receive FMT from responders and non-complete responders. Tumor growth can be monitored and survival rate can be recorded. In addition, we can utilize IFN-g KO (C.129S7 (B6)-Ifng^{tm1Ts}/J (IFN-γ^{KO}) mice to examine the modulatory effects of FMT and to determine the correlation between Th I immune response and gut microbiome. Similarly, we can examine the FMT efficacy in FCR receptor KO mice (C.129P2 (B6)-Fcer1g^{tm1Rav} N12) mice) to determine the correlation between antibody mediate immune response and gut microbiome.

c) MYD88 KO Mice Model:

To determine whether the systemic immune response following FMT characterized by specific microbial signatures from responders is mediated through TLR signaling activation, we can utilize MYD88 KO mice to verify the role of TLR signaling. HER2^{POS} TUBO tumor bearing MYD88 knockout mice or 4T1/EO771 tumor bearing MYD88 knockout mice can be divided into following groups and can receive the following treatment conditions respective of tumor models: 1) control; 2) DC1 alone; 3) FMT from responders; 4) LPS+CPG ODN; 5) LPS+CPG ODN. The tumors can be measured for tumor growth and survival.

d) Cross-Transfer of FMT Between Different Subtypes of BC Models:

To investigate whether the microbiome characterized by particular microbial signatures from the complete responders of HER2^{pos} or TNBC murine model benefit both HER2^{POS} and TNBC tumor bearing mice, we can utilize 4T1 and EO771 in Balb/C or C57BL/6 mice. When tumors are palpable, mice can be randomized as described above. Tumor bearing mice can receive FMT from complete responders and mice immune to subsequent rechallenge with HER2^{pos} TUBO cells or TNBC cells. These studies can address the crucial role of identifying universal favorable microbial signatures in mediating tumor regression irrespective of the tumor subtypes.

e) Flow Cytometry and Immunophenotyping:

The mechanism of anti-tumor activity of the specific microbial signatures in different breast cancer subtypes can be investigated. We can perform a comprehensive analysis of T cells and suppressor cell populations in the spleen, lymph nodes (LNs), and tumors as described herein.

f) Molecular Mechanism of Action:

The tumor tissue lysates can be examined for the expression of HER2 dependent signaling cascades (HER2, ERK1/2, PI3K, AKT, mTOR, and p38 MAPK), TLR signaling (TLR4, TLR9 and MyD88), Th1 cytokine downstream signaling cascades (STAT1, STAT3 and STAT5), EMT and stem cell signaling markers (Wnt4, N-cadherin, E-cadherin, β-catenin, vimentin, HIF-1α, ALDHIA1, Slug and Snail), senescence marker proteins (p15/INK4B and p16/ARF) and apoptosis marker proteins (caspase-3 and cleaved-caspase 3) by western blot. In addition, paraffin-embedded tumor tissues can be stained for HER2, Ki-67, VEGF, VEGFR2 and HIF-1alpha. The single cell suspension prepared from tumor tissue of experimental groups can be examined for senescence stained by beta-gal blue staining.

### 9. Example 9: Determine Whether the Microbial Signature or Metabolomic Changes can be Manipulated to Enhance Complete Immunologic Responses in Both Preventive and Therapeutic Mammary Carcinoma Models to Immunotherapies and the Mechanism by which this Occurs

a) Investigate Whether FMT from Responders can Improve DC1 Efficacy Alone or in Combination with Other Therapies in neuT Mice Model in Both Preventive and Therapeutic Settings

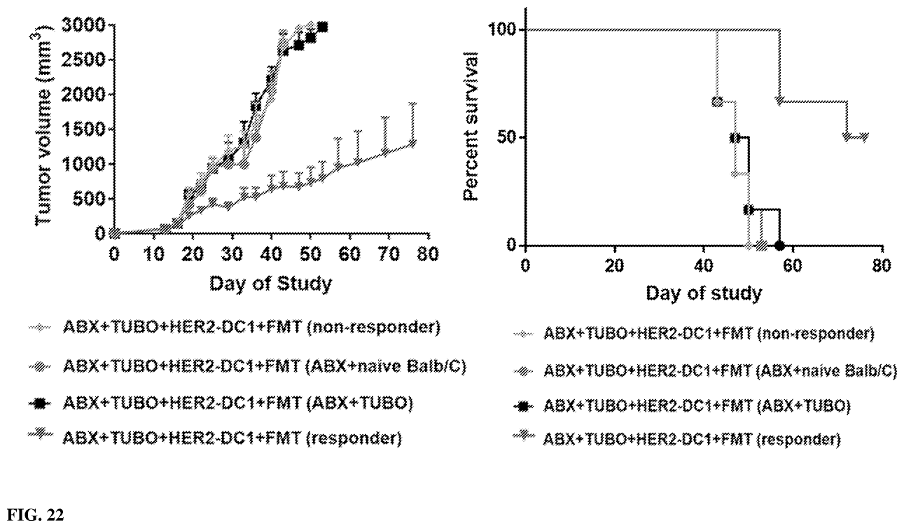

Here, we can examine the role of specific favorable microbial signatures from complete responders in a Transgenic HER2 mammary cancer model (Balb/C Neu T). The key features of this model are each of their ten mammary glands develops an independent carcinoma that slowly progresses from microscopic lesions to invasive tumors. Since this model mimics some of the most critical features of human disease, this is a useful model to investigate the preventive and therapeutic efficacy. MRI images for week 8 showed no microscopic tumor growth in all 10 mammary glands of neu T mice. Week 10-11 and week 12-13 MRI images showed evidence for the spontaneous mammary carcinoma development. At the age of week 16, nearly 7 to 8 mammary glands detected positive for mammary tumor growth (FIG. 10).

Neu T mice can be randomized into following treatment groups. 1) control; 2) HER2-DC1; 3) antibiotics+HER2-DC1+ FMT from neu T mice; 4) HER2-DC1+ FMT from responders; 5) HER2-DC1+FMT from responders+anti-SEMA4D; 6) HER2-DC1+ FMT from responders+anti-HER2; 7) HER2-DC1+ FMT from responders+CDK inhibitors; 8) HER2-DC1+ FMT from responders+Immune checkpoint inhibitors; 9) antibiotics+HER2-DC1+ FMT from responders; 10) antibiotics+HER2-DC1+ FMT from responders+anti-SEMA4D; 11) antibiotics+HER2-DC1+ FMT from responders+anti-HER2; 12) antibiotics+HER2-DC1+ FMT from responders+CDK inhibitors; 13) antibiotics+DC1+ FMT from responders+Immune checkpoint inhibitors. All monotherapy or FMT or antibiotics treatments or combination therapy can be administered as detailed herein. For preventive settings, treatments can be initiated in neu T mice at the age of 8 weeks old after verifying no microscopic mammary tumor mass in all 10 mammary glands by MRI. Intramammary gland delivery of HER-DC1 vaccine can be carried out for respective groups guided by ultrasound. For therapeutic settings, all monotherapy or FMT or antibiotics or combination therapy can be started in neu T mice at the age of 11 weeks old when mice can develop microscopic tumor masses verified by MRI. Fecal samples can be collected at various time points and screened for microbial composition/species richness as detailed herein. Spontaneous mammary carcinoma development can be monitored by MRI at the regular intervals. After the completion of the treatments, mice can be sacrificed from both preventive and therapeutic settings.
b) Efficacy of Immune Based Therapy in Combination with FMT from Responder in Germ Free Mice Model

Germ free mice (Balb/C or C57BL/6 mice) can be injected with either HER2^{POS} TUBO cells or with 4T1/EO771 TNBC cells subcutaneously. When mice develop palpable tumors, they can be randomized to following groups respective of tumor types: 1) control; 2) HER2DC1; 3) HER3-DC1; 4) HER2/HER3-DC1+FMT from complete responders; 4) HER2/HER3-DC1+FMT from non-complete responders. All treatments can be carried out as detailed herein. Tumor growth can be monitored every 2-3 days and survival rate can be documented. Fecal samples can be collected from both experiments.

c) Analysis of Immune Responses and Immune Subsets

Anti-HER2/HER3 Th1 immune responses can be measured by co-culturing splenocytes or lymph node cells with class II HER2 or HER3 peptides for 48-72 h. Culture supernatants can be collected and analyzed for IFN-γ by standard ELISA. We can measure various immune subsets including CD4 T cells, CD8 T cells, T reg cells, B cells, natural killer (NK) cells, NKT cells, dendritic cells, macrophages and myeloid derived suppressor cells by flow cytometry. The intestine, proximal, and distal segments of the colon can be examined for inflammatory cell invasion into the epithelium. In addition, paraffin-embedded tumor tissues and intestine can be stained for CD4, CD8, F4/80, CD19 and DX-5 markers.

d) Role of Microbial Metabolomics in Response to Immune Based Therapy

Recent studies on gut microbiota are increasingly revealing various bacterial metabolites and bacterial components that can modulates various cellular signaling pathways and regulates immune system. Based on these evidences, we can examine the status of various bacterial metabolites in response to immune based therapy in combination with FMT in HER2+ BC and TNBC preclinical models. We can look at broad range of bacterial components and metabolites bacterial lipopolysaccharides (LPS), peptide polysaccharide A (PSA), flagellin, sphingolipids, α-galactosylceramide, mycolic acid, glucose monomycolate, trehalose-6,6-dimycolate, short-chain fatty acids, cholic acid, chenodeoxycholic acid, propionate, butyrate, hexanoate, benzoate, niacin, urolithin-A, equol, indole, 8-prenylnaringenin, commendamide, protocatechuic acid, trimethylamine-N-oxide, lactocillin, linaclotide, indolepropionic acid, tryptamine, terpenoid deoxycholic acid, corynebactin, tilivalline, mutanobactin, mycolactone, peptide microcin E492 and lipid A. To address this, we can utilize HER2 and TNBC models as described in herein (Germ free mice or Balb/c or C57BL/6). Untargeted whole microbial metabolome can be measured in controls, DC1+FMT from responders compared to DC1+FMT from non-responders, control+immature DCs (iDCs), iDCs+FMT from responders and iDCs+FMT from non-responders. Metabolon© can analyze stool samples and provide reports of raw and median-scaled metabolite abundances. For each metabolite, differences in median-scaled abundance across groups can be assessed with univariate ANOVA. Metabolomic signatures that differentiate each group can be identified with sPLS-DA. A multiomics integrative analysis can be performed with Data Integration Analysis for Biomarker discovery using Latent cOmponents (DIABLO), an extension of Generalized Canonical Correlation Analysis for multiomics signature discovery. Random Forest classifiers can be used to further identify associations across study outcomes and assess whether observed changes in microbial/metabolomic composition are predictive of group membership. The relative importance of each predictor in high performing classifiers, as determined through consideration of accuracy, precision, and recall, can be used as a secondary measure of significance. Minimal, high performing models can be made publicly available.

e) DC1 Vaccine in Combination with TLR4 and TLR9 Agonist Therapy

The conserved components of gut bacteria such as lipopolysaccharide (LPS) and CpG oligodeoxynucleotides (CpG ODN) recognizes toll-like receptor 4 (TLR4) and TLR9 that are expressed on DC. Activation of TLR4 and TLR9 signaling promotes maturation and improves the function of DC leading to strong anti-tumor immune response. In order to examine whether the enhanced anti-tumor response of DCI vaccine following FMT from responders is mediated through bacterial metabolomics dependent activation of TLR 4 and 9 signaling in DC, HER2^{POS} TUBO bearing mice or 4T1/EO771 TNBC bearing mice can be divided in to four groups respective of tumor models and can receive following treatment conditions: 1) control; 2) LPS (TLR4 agonist) and CPG ODN (TLR9 agonist); 3) -DC1 vaccine alone; 4) DC1 vaccine in combination with LPS+CPG ODN. At the end of the treatments, mice can be sacrificed to collect tumors, spleen, lymph node, whole intestine and blood for various in vitro functional assays and multicolor flow cytometry staining as detailed herein.

f) Molecular Mechanism of Action

The tumor tissue lysates can be examined for the expression of different dependent signaling cascades as described herein.

### 10. Example 10: Determine in Human Breast Cancer Patients Whether the Microbial Signature is Altered in Complete Responders to Either Immune Based or Standard Neoadjuvant Therapy

Having identified a microbial and metabolomic signature that drives an anti-tumor immune response in preclinical models we can attempt to identify similar signatures in patients with breast cancer undergoing neoadjuvant therapy. The data in murine tumor models indicates that mice which develop complete response to immunotherapy demonstrate changes in their gut microbiome compared to non-responders and that transfer of the microbiome to tumor bearing mice in combination with partially effective immunotherapy resulted in complete responses in most mice (FIGS. 1 and 9). We can therefore characterize the stool microbiome and targeted microbial metabolome, based on the metabolic findings herein.

### Approach:

a) Population

130 BC patients (pts) receiving immune based or standard neoadjuvant therapy using 16S rRNA gene sequencing, selected metagenomic whole genome shotgun (WGS) sequencing, and targeted metabolomics. We can collect stool samples at several time points: Pre-treatment then post immunotherapy and after any additional standard chemotherapy treatment. Analysis of composition of microbiomes (ANCOM) can be used to identify differential abundant amplicon sequence variants (ASVs). We can correlate the stool microbiome composition in pCR and non-pCR to immune based or standard neoadjuvant therapy. A similar approach to metabolome as is described herein can be employed. Patients can have TNBC or HER2^{pos} BC (100 pts). A subpopulation of 30 ER+HER2− breast cancer patients undergoing neoadjuvant therapy can be collected as controls. We anticipate with TNBC and HER2 populations about 40-50% can have pCR to standard or standard therapy with immunotherapy. Trastuzumab and pertuzumab are considered immunotherapies as are vaccines and checkpoint inhibitors. The 30 ER+ pts can be evaluated pre-therapy for comparison of microbiome pre-treatment with TNBC and HER2.

b) Endpoints

The primary objective of this proposal is to determine whether the microbial signature is predictably altered in patients with a pCR to either immune-based or standard neoadjuvant chemotherapy for BC.

c) Fecal Collection

Patients can be provided with the study materials, a copy of the signed informed consent, and an at-home fecal collection kit (OMNIgene GUT kit) in clinic or by mail. The Research Coordinator can explain the fecal collection process with each participant, in the clinic or over the phone. Three brief questions can be included with the at-home stool collection kit: recent antibiotic use, current prebiotic/probiotic use, and type of diet. Two days prior to the first research blood draw, the Research Coordinator can call to remind the patient to collect their fecal specimen within 72 hours of their visit and bring the specimen with them to the Clinical Research Unit (CRU), where study blood draws are performed. On the day of their blood draw, the patient can bring the fecal sample with them to the CRU, where the Research Coordinator can retrieve the sample. At subsequent study visits, participants can be asked to provide a fecal sample within 72 hours of their next appointment and bring the fecal sample with them to their CRU appointment. At all 3 study visits, the Research Coordinator can retrieve the fecal specimen from the patient, verify all specimen labels, and enter the specimen information into a secure database. The prepackaged specimen tubes with liquid preservatives can be vortexed slightly at room temperature to homogenize and stored at −80° C. in the Czerniecki lab. Fecal samples can be screened for microbial composition/species richness by 16S ribosomal RNA (rRNA) gene-based sequencing of various bacterial gene amplicons and metagenomics whole genome shotgun DNA sequencing as described herein. The routine 16SrRNA gene sequencing is done using the Illumina MiSeq, but the WGS requires a NexSeq or HISeq Illumina instrument and samples can be prepared in the Groer laboratory and sequenced. Microbial Signature analysis can be performed as described herein.

### 11. Example 11: Determine Whether Specific Bacterial Species Identified in Human Subject that Achieve a pCR can Mediate an Anti-Tumor Response in Murine Mammary Carcinoma in a Germ-Free Mice Model

a) FMT from Responders and Non-Responders of HER2 BC Patients or TNBC Patients after Immune Based Therapy/Standard Care of Therapy

Germ-free mice can be injected with HER2^{POS} TUBO cells at the MFP or 4T1/EO771 TNBC cells subcutaneously. When tumors are palpable, mice can be randomized to experimental groups with following treatment conditions respective of tumor models: 1) control; 2) FMT from responders; 3) FMT from non-responders; 4) HER2 or HER3-DC1; 5) HER2 or HER3-DC1+FMT from responders; 6) HER2 or HER3-DC1+FMT from non-responders. Stool samples can be suspended in sterile PBS and filtered through 100 μm strainer. 100 ul of cleared fecal suspension can be gavaged into mice. The tumor growth can be monitored and measured for survival. Fecal samples can be collected from experimental mice and screened from microbial compositions and metabolomics as detailed herein. In addition, tumors, spleen, lymph node, intestine and blood can be collected for in vitro functional assays as described herein. Tumor tissue samples can be examined for HER2 dependent signaling cascades, TLR signaling, Th1 cytokine downstream signaling cascades, EMT markers, stem cell signaling markers, senescence marker proteins, apoptosis marker proteins and angiogenesis markers as detailed herein.

1. An anti-cancer combination therapy comprising i) at least one oncodriver pulsed dendritic cell and ii) a fecal microbial transplant (FMT) from a pathologic complete response (pCR) donor or a cyclin-dependent kinase (CDK) inhibitor.

2. The anti-cancer combination therapy of claim 1, wherein the oncodriver is selected from the group consisting of human epidermal growth factor receptor (HER) 1 (HER1), HER2, HER3, EGFR, c-MET, B-Rapidly Accelerated Fibrosarcoma (BRAF), KIT, Androgen Receptor (AR), Estrogen Receptor (ER), KRAS, TP53, and APC.

3. The anti-cancer combination therapy of claim 1, wherein the oncodriver pulsed dendritic cell is activated with IL-12 prior to administration.

4. The anti-cancer combination therapy of claim 1, wherein the FMT comprises enriched Anaerosporobacter.

5. The anti-cancer combination therapy of claim 1, wherein the CDK inhibitor comprises abemaciclib, ribociclib, palbociclib, trilaciclib, or taxol.

6. A method of treating a cancer in a subject comprising administering the anti-cancer combination therapy of claim 1.

7. The method of treating a cancer of claim 1, wherein the wherein the oncodriver pulsed dendritic cell is administered intratumorally.

8. A method of treating a cancer in a subject comprising administering to the subject i) an oncodriver pulsed dendritic cell and ii) a fecal microbial transplant (FMT) from a pathologic complete response (pCR) donor or a cyclin-dependent kinase (CDK) inhibitor.

9. The method of treating a cancer of claim 8, wherein the oncodriver is selected from the group consisting of human epidermal growth factor receptor (HER) 1 (HER1), HER2, HER3, EGFR, c-MET, B-Rapidly Accelerated Fibrosarcoma (BRAF), KIT, Androgen Receptor (AR), Estrogen Receptor (ER), KRAS, TP53, and APC.

10. The anti-cancer combination therapy of claim 8, wherein the FMT comprises enriched Anaerosporobacter.

11. The anti-cancer combination therapy of claim 8, wherein the CDK inhibitor comprises abemaciclib, ribociclib, palbociclib, trilaciclib, or taxol.

12. The method of treating a cancer of claim 8, wherein the dendritic cells are removed from the subject and pulsed with oncodriver ex vivo.

13. The method of treating a cancer of claim 8, wherein the pulsed dendritic cells are administered intratumorally.
